(19) United States
(12) Patent Application Publication
(10) Pub. No.: US 2009/0326031 A1
(43) Pub. Date: Dec. 31, 2009

(54) ANTIVIRAL USE OF CATIONIC SURFACTANT

(75) Inventors: Xavier Rocabayera Bonvila, Barcelona (ES); Sergi Figueras Roca, Barcelona (ES); Roger Segret Pons, Barcelona (ES)

Correspondence Address: RENNER OTTO BOISSELLE & SKLAR, LLP, 1621 EUCLID AVENUE, NINETEENTH FLOOR, CLEVELAND, OH, 44115, US

(73) Assignee: NOVACYT, Velizy Villacoublay (FR)

(21) Appl. No.: 12/375,774

(22) PCT Filed: Aug. 3, 2006
(86) PCT No.: PCT/EP2006/065035

§ 371 Date: Mar. 26, 2009

(30) Foreign Application Priority Data
Jul. 31, 2006 (FR) 0607005

(51) Int. Cl. A61K 31/221; A61K 31/4164; A61P 31/12

(52) U.S. Cl. 514/400; 514/551

(57) ABSTRACT

Cationic surfactants derived from the condensation of fatty acids and esterified dibasic amino acids, such as from lauric acid and arginine, in particular the ethyl ester of the lauramide of the arginine monohydrochloride (LAE), may be used for the protection against the growth of the microorganisms. The cationic surfactants of this type are also effective against virus infections. Addition of LAE to cultures of Herpes virus type 1 Vaccinia virus and bovine parainfluenzae 3 virus leads to nearly complete reduction of the virus organisms in these cultures, such effects being observed after 5 and 60 minutes.

The present application relates to a novel use of cationic surfactants.

Cationic surfactants are well-known in the art for a variety of different applications.

Cationic surfactants derived from lauric acid and arginine, in particular the ethyl ester of the lauramide of the arginine monohydrochloride, hereafter named LAE, may be used for the protection against the growth of the microorganisms. The chemical structure of LAE is described in the following formula (1):

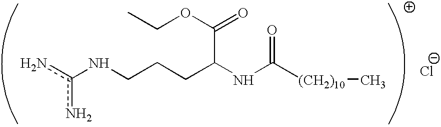

The preparation of this product has been described in Spanish patent application ES-A-512643.

The metabolism of the above cationic surfactant of formula (1) in rats has been studied, these studies have shown a fast absorption and metabolisation into naturally-occurring amino acids and the fatty acid lauric acid, which are eventually excreted as carbon dioxide and urea. Toxicological studies have demonstrated, that LAE is completely harmless to animals and humans.

Therefore, LAE and related compounds are particularly suitable to be used in the preservation of all perishable food products. LAE and related compounds are equally suitable for use in cosmetic products.

This compound is remarkable for its inhibitory action over the proliferation of different microorganisms, such as bacteria, fungi and yeasts. The minimum inhibitory concentrations of LAE are shown in the following table 1.

TABLE 1

| | | M.I.C. |
|---|---|---|
| Kind | Microorganism | (ppm) |
| Gram + Bacteria | Arthrobacter oxydans ATCC 8010 | 64 |
| | Bacillus cereus var mycoide ATCC 11778 | 32 |
| | Bacillus subtilis ATCC 6633 | 16 |
| | Clostridium perfringens ATCC 77454 | 16 |
| | Listeria monocytogenes ATCC 7644 | 10 |
| | Staphylococcus aureus ATCC 6538 | 32 |
| | Micrococcus luteus ATCC 9631 | 128 |
| | Lactobacillus delbrueckii ssp lactis CECT | 16 |
| | 372 | |
| | Leuconostoc mesenteroides CETC 912 | 32 |
| Gram − Bacteria | Alcaligenes faecalis ATCC 8750 | 64 |
| | Bordetella bronchiseptica ATCC 4617 | 128 |
| | Citrobacter freundii ATCC 22636 | 64 |
| | Enterobacter aerogenes CECT 689 | 32 |
| | Escherichia coli ATCC 8739 | 32 |
| | Escherichia coli 0157H7 | 20 |
| | Klebsiella pneumoniae var pneumoniae | 32 |
| | CECT 178 | |
| | Proteus mirabilis CECT 170 | 32 |
| | Pseudomonas aeruginosa ATCC 9027 | 64 |
| | Salmonella typhimurium ATCC16028 | 32 |
| | Serratia marcenses CECT 274 | 32 |
| | Mycobacterium phlei ATCC 41423 | 2 |
| Fungi | Aspergillus niger ATCC14604 | 32 |
| | Aureobasidium pullulans ATCC 9348 | 16 |
| | Gliocadium virens ATCC 4645 | 32 |
| | Chaetonium globosum ATCC 6205 | 16 |
| | Penicillium chrysogenum CECT 2802 | 128 |
| | Penicillium funiculosum CECT 2914 | 16 |
| Yeast | Candida albicans ATCC 10231 | 16 |
| | Rhodotorula rubra CECT 1158 | 16 |
| | Saccharomyces cerevisiae ATCC 9763 | 32 |

The use of the invention relates to cationic surfactants derived from the condensation of fatty acids and esterified dibasic amino acids, according to the following formula (2):

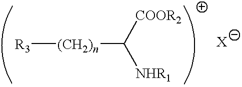

where:
X⁻ is Br⁻, Cl⁻, HSO₄⁻, a counter ion derived from an organic or inorganic acid, or an anion on the basis of a phenolic compound;
R₁: is a linear alkyl chain from a saturated fatty acid or hydroxyacid from 8 to 14 atoms of carbon bonded to the α-amino acid group through an amidic bond;
R₂: is a linear or branched alkyl chain from 1 to 18 carbon atoms or an aromatic group;

### R₃: is

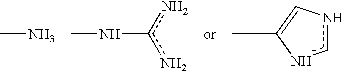

and n can be from 0 to 4.

The organic acids which may be the source of the counter ion X⁻ can be citric acid, lactic acid, acetic acid, fumaric acid, maleic acid, gluconic acid, propionic acid, sorbic acid, benzoic acid, carbonic acid, glutamic acid or other amino acids, lauric acid and fatty acids such as oleic acid and linoleic acid, whereas the inorganic acids can be phosphoric acid, nitric acid and thiocyanic acid.

The phenolic compound which may be the basis of the anion X⁻ is for instance butylated hydroxyanisole (BHA) and the related butylated hydroxytoluene, tertiary butyl hydroquinone and parabens such as methylparaben, ethylparaben, propylparaben and butylparaben.

The most preferred compound of the above class of compounds is LAE.

It is preferred to dissolve the compound directly before use in one of the following preferred solvents of food grade: water, ethanol, propylene glycol, isopropyl alcohol, other glycols, mixtures of glycols and mixtures of glycols and water. If the treatment shall be performed at a specific pH value the use of a corresponding buffer solution may be recommendable. On the other hand the compound can be easily used as a solid. Surfaces which shall be protected by solid preparations are for instance the surfaces of food products or cosmetics.

For the cationic surfactants of the above formula (2) the antibacterial activity and the biological activity against other microorganisms such as fungi and yeasts is well documented. An activity of the cationic preservatives against viruses has not been described.

Safe and effective products for the treatment of viral infections are urgently and constantly needed. World-wide outbreaks of virus infections illustrate the complexity of effective treatments. Recently the outbreak of avian flu led to an urgent search for the optimum tools to limit the spreading of the disease.

Avian influenza, nowadays also known as avian flu, is a bird disease caused by influenza A viruses, essentially of haemagglutinin subtypes H5 or H7, which belong to the family Orthomyxoviridae. Within the same group of viruses the virus responsible for the respiratory disease called influenza or flu is found.

Parainfluenza viruses, included in subfamily Paramyxovirinae, with the family of the Paramyxoviridae, include viruses affecting humans as a well as animals. Virions are 150 nm in diameter, pleomorphic, with a lipid envelope derived from the host cell membrane and with projections of 10-14 nm in length. Its genome contains 7-8 segments of linear, negative sense, single stranded RNA, resulting in a genome size of 10,000 to 14,600 nucleotides.

Furthermore, Paramyxoviridae and Orthomyxoviridae share special relationships with respect to the biological properties of the envelope glycoproteins. As seen in the following table 2, Paramyxoviridae and Orthomyxoviridae do not display major differences in their protein, lipid and carbohydrate content.

TABLE 2
Protein, lipid and carbohydrate content of members of families
Paramyxoviridae and Orthomyxoviridae.

| Content (%) | Paramyxoviridae | Orthomyxoviridae |
|---|---|---|
| Proteins | 70-74 | 70-77 |
| Lipids | 20-25 | 18-37 |
| Carbohydrates | 6 | 5-8 |

The term parainfluenza was originally chosen, because some of the disease symptoms are highly similar to those of influenza and both virus types display hemagglutination and neuraminidase activities.

Influenza virus and parainfluenza virus display the same sensitivity against external influences such as heat.

It is a belief in the art, that parainfluenza virus is a good experimental model for the determination of the biological activity of test compounds against influenza virus.

It is the unexpected result of the investigations made by the inventors, that the cationic surfactants of above formula (2) displayed a surprising, remarkably strong antiviral activity. Until now this class of compounds was known for its antimicrobial action and an antivirus action could not be expected.

The antiviral activity could be observed against viruses of Vaccinia, Herpes simplex and bovine parainfluenzae virus types. Particularly strong was the action against viruses of the Herpes and bovine parainfluenzae virus types, in which types a short contact time of 5 minutes was sufficient for achieving maximum effects. This may be considered as the particularly surprising aspect of the present invention, of achieving the effect against the viruses after such a short time.

There is a similarity between the virus types which have turned out to be most sensitive to the antiviral action of the cationic surfactants such as the Vaccinia, Herpes simplex and bovine parainfluenzae virus types and a virus such the adenovirus which is the agent which is responsible for AIDS. The cationic surfactants which have been investigated by the present inventors are found to be effective against human immunodeficiency virus I as well.

The present invention relates to the use of the cationic surfactants of formula (2) as treating any kind of products which are affected by a virus contamination. Such products may for instance be warm dishes or warm liquids. The products to be treated may be any kind of equipment which is used in the handling of animals which are infected with virus. In an even broader sense the products to be treated can be the facilities where animals are kept, or parts of the natural environment such as the ground surface or water reservoirs.

The present invention furthermore relates to the administration of the cationic surfactants of formula (2) to animals or human beings directly, for prophylactic or therapeutic treatment of virus diseases.

The cationic surfactants of formula (2) may be applied as a solution. This is the easy and suitable manner of treating the surface of the ground, cars and people. For other applications it may be more suitable to apply the cationic surfactant as a solid, which may be equally effective.

The treatment of products in order to avoid any kind of virus infection might involve the presence of a concentration of the cationic surfactants of the formula (2), more in particular according to the preferred embodiment of LAE, of around 2 to 20,000 ppm of the product to be protected, preferably a concentration of 100 to 10,000 ppm, and more preferably 200 to 2,000 ppm. This is a similar concentration as has been described for achieving the microbiocidal action. Products to be treated with the above-indicated range of concentrations of the cationic surfactants are for instance food products or cosmetics.

The treatment of surfaces which are infected with viruses, such as the surface of food preparations, the surface of cosmetics, ground surface, the surface of any kind of vehicles, and the surface of any equipment used in the handling of animals infected with virus, requires the presence of the cationic surfactants of the formula (2), more in particular according to the preferred embodiment of LAE, at a concentration of a level which is sufficient to achieve the wanted antiviral action at such surfaces. Such level of concentration would be expected in the range of 2 to 20,000 ppm, more preferred 100 to 10,000 ppm and even more preferred 200 to 2,000 ppm, containing the cationic surfactant of claims, according to the preferred embodiment containing LAE. These concentrations are given in terms of the concentration of a solution containing the cationic surfactant which is applied to the surfaces to be treated. If surfaces are treated with a solid preparation of the cationic surfactant of formula (2), the amount which is applied shall be such that the amount of the cationic surfactant of formula (2) shall be in the range of 0.01 to 100 mg/dm², preferably an amount of 0.5 to 50 mg/dm², and more preferably an amount of 1 to 10 mg/dm².

The treatment of liquid preparations such as drinking fluids or natural sources of water such as lakes or ponds requires the presence of the cationic surfactants of the formula (2), more in particular according to the preferred embodiment of LAE, at a concentration of a level which is sufficient to achieve the wanted antiviral action in the drinking fluid or water. Such level of concentration would be expected in the range of 0.2 to 20,000 ppm, more preferred 2 to 15,000 ppm, even more preferred 100 to 10,000 ppm and most preferred 200 to 2,000 ppm, containing the cationic surfactant of formula (2), according to the preferred embodiment containing LAE. These concentrations are provided in terms of the concentration of the cationic surfactant in the liquid or the water to be treated.

The treatment of animals or humans implies the administration of the cationic surfactants in a manner which is suitable for absorption of compounds used according to the invention. The compounds can be administered orally, parenterally (including intraperitoneal, subcutaneous and intramuscular injections) or externally (topically, such as rectal, transdermal, by instillation and transnasal). The preparation to be administered may have the form of a conventional pharmaceutical preparation such as capsules, microcapsules, tablets, enteric coated agents, granules, powder, pills, ointments, suppositories, suspensions, syrups, emulsions, liquids, sprays, inhalants, eye drops and nose drops.

The above-mentioned pharmaceutical preparations can be produced according to conventional methods using various organic or inorganic carriers conventionally used for the pharmaceutical formulation of preparations, such as excipients (such as sucrose, starch, mannit, sorbite, lactose, glucose, cellulose, talc, calcium phosphate, calcium carbonate), binders (such as cellulose, methyl cellulose, hydroxymethyl cellulose, polypropylpyrrolidone, gelatin, Arabic gum, polyethylene glycol, sucrose, starch), disintegrants (such as starch, carboxymethyl cellulose, hydroxypropyl starch, sodium hydrogen carbonate, calcium phosphate, calcium citrate), lubricants (such as magnesium stearate, aerosyl, talc, sodium lauryl sulphate), corrigents (such as citric acid, menthol, glycine, orange powder), preservatives (sodium benzoate, sodium bisulfite, methylparaben, propylparaben), stabilizers (such as citric acid, sodium citrate, acetic acid), suspending agents (methyl cellulose, polyvinylpyrrolidone, aluminium stearate), dispersing agents (such as hydroxypropylmethyl cellulose), diluents (such as water), base waxes (such as cacao butter, white petrolatum, polyethylene glycol) and other suitable ones.

The dose of the cationic surfactant according to the use of the present invention shall be determined by the dose required for achieving the wanted prophylactic or therapeutic effect. A usual dose shall be 0.1 mg/kg to 10 mg/kg for oral or parenteral administration. A usual dose in humans may be a unit dose of 0.1 to 1000 mg per individual, more preferable 0.5 to 500 mg per individual. This dose may be administered 1 to 4 times per day, depending on the severity of the symptoms. A usual dose in animals may be 0.1 to 100 mg per dose, preferred 0.5 to 50 mg per dose.

The effect of the use of the invention is illustrated through the following examples.

In the following experiments the reduction factor has been calculated.

This reduction factor is calculated according to the following formula

$${10}^{{R}^{′}} = \frac{{v}^{′} \cdot {10}^{{a}^{′}}}{{v}^{″} \cdot {10}^{{a}^{″}}}$$

In this formula:

R′ presents the reduction factor,
v′ represents the volume of the sample of the test compound,
a′ is the virus concentration in the added sample (¹⁰log value),
v″ is the volume of the final sample,
a″ is the virus concentration after treatment (¹⁰log value).

In the following experiments the clearance factor has been calculated. The clearance factor is calculated according to the following formula:

$${10}^{CL} = \frac{{10}^{{q}^{′}}}{{10}^{{q}^{″}}}$$

In this formula:

CL is the elimination factor (clearance factor),
q′ is the initial concentration of virus (¹⁰log value),
q″ is the final concentration of virus (¹⁰log value).

### EXAMPLE 1

### The Activity of LAE was Investigated Against the Virus Bovine Parainfluenzae 3 Virus

### Medium for Cell Cultivation

Eagle minimum essential medium (EMEM) was obtained from Earle (ICN Flow, Rf. 11-100-24), was supplemented with

10% of fetal bovine serum (Biowhittaker, Ref. D14-810F),
2 nM of glutamine (Biowhittaker, Ref. 17-605E),
non-essential amino acids (Biowhittaker, Ref. 13-114E),
100 units of penicillin,
100 μg of streptomycin/ml and
1 mM of sodium pyruvate.
LAE (CAS 60372-77-2) Lot No. 10234 with a LAE Content of 88.7%.

The investigated strain of the parainfluenza bovine type 3 was SF-4 (ATCC VR-281).

### Experimental Conditions:

LAE was investigated at a concentration of 200 ppm.

All experiments were performed at room temperature.

8 ml of deionized water was mixed with 1 ml of the virus suspension. 1 ml of LAE solution (containing 2000 ppm of LAE) was added, leading to a final LAE concentration of 200 ppm.

After 5 and 60 minutes samples for determination were taken. The design of the experiment allowed for conclusions of the effectivity of LAE after contact times of 5 and 60 minutes.

The results are displayed in the following table 3.

TABLE 3

| Virus concentration | |
|---|---|
| Before addition of LAE | 8.09 ± 0.33 |
| 60 min after addition of LAE | ≦1.63 |
| Reduction factor | ≧6.46 ± 0.32 |
| Clearance factor | ≧6.29 ± 0.32 |

After treatment with LAE for 60 minutes no remaining virus organisms were detected. Similar results were achieved after 5 minutes.

The number of virus particles in the experiment were determined by the quantitative assay TCID50 (infectious dose of cultured tissue).

### EXAMPLE 2

### Activity of LAE Against Herpes Virus Type 1

The virus which was investigated was the type Simplex virus of the subfamily Alphaherpesvirinae in the family Herpesviridae.

The conditions for the determination are similarly chosen according to the ones of example 1.

LAE was investigated at a concentration of 200 ppm.

The results are displayed in the following table 4.

TABLE 4

| Virus concentration | |
|---|---|
| Before addition of LAE | 7.94 ± 0.37 |
| 60 min after addition of LAE | ≦1.70 |
| Reduction factor | ≧6.18 ± 0.36 |
| Clearance factor | ≧6.24 ± 0.37 |

After treatment with LAE for 60 minutes no remaining virus organisms were detected. Similar results were achieved after 5 minutes.

### EXAMPLE 3

### Activity of LAE Against Vaccinia Virus

The virus which was investigated was the type Orthopoxvirus from the family Poxviridae.

The family of the Poxviridae covers complex viruses having a complex structure with a nucleus containing viral DNA (double chain with 170-250 kbp).

LAE was investigated at a concentration of 200 ppm.

The results are displayed in the following table 5.

TABLE 5

| Virus concentration | |
|---|---|
| Before addition of LAE | 5.94 ± 0.34 |
| 60 min after addition of LAE | 2.40 |
| Reduction factor | 3.92 ± 0.36 |
| Clearance factor | 3.54 ± 0.34 |

After treatment with LAE for 60 minutes a substantial decrease of the number of living Vaccinia virus organisms was detected. A great reduction of the number of living microorganisms was also observed after treatment for 5 minutes.

1. Use of a cationic surfactant derived from the condensation of fatty acids and esterified dibasic amino acids, according to the following formula (2): 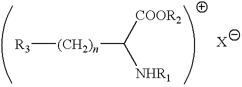
where:
X⁻ is Br⁻, Cl⁻, or HSO₄⁻, a counter ion derived from an organic or inorganic acids, or an anion on the basis of a phenolic compound;
R₁: is a linear alkyl chain from a saturated fatty acid or hydroxyacid from 8 to 14 atoms of carbon bonded to the α-amino acid group through an amidic bond,
R₂: is a linear or branched alkyl chain from 1 to 18 carbon atoms or an aromatic group,
R₃: is 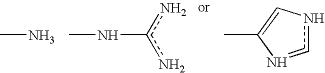
and n can be from 0 to 4,
for the treatment of virus infections.

2. The use of claim 1, for the manufacture of a medicament for the treatment of virus infections in animals or human beings.

3. The use of claim 2, whereby the cationic surfactant is administered orally, parenterally (including intraperitoneal, subcutaneous and intramuscular injections) or externally (topically, such as rectal, transdermal, by instillation or transnasal).

4. The use of claim 3, in which the cationic surfactant of the formula (2) is the ethyl ester of the lauramide of the arginine monohydrochloride (LAE).

5. The use of claim 2, for the treatment of a disease caused by an influenza virus.

6. The use of claim 5, in which the influenza virus is the virus causing avian influenza.

7. The use of claim 2 for prophylactic application to animals or human beings.

8. The use of claim 1, for the treatment of surfaces which are infected with viruses, such as the surface of food preparations, the surface of cosmetics, ground surface, the surface of any kind of vehicles, and the surface of any equipment used in the handling of animals infected with virus.

9. The use of claim 1, for the treatment of liquid preparations such as drinking fluids or natural sources of water.

10. The use of claim 1, for the treatment of food preparations.

11. The use of claim 1, for the treatment of cosmetic preparations.

12. The use of claim 1, in which the virus is a Vaccinia, Herpes simplex and bovine parainfluenzae virus type virus.
